(19) United States
(12) Patent Application Publication
(10) Pub. No.: US 2017/0136226 A1
(43) Pub. Date: May 18, 2017

(54) VASCULAR ACCESS DEVICE AND METHOD OF USE THEREOF

(71) Applicant: COOK MEDICAL TECHNOLOGIES LLC, BLOOMINGTON, IN (US)

(72) Inventor: Jeffry S. Melsheimer, Springville, IN (US)

(73) Assignee: COOK MEDICAL TECHNOLOGIES LLC, BLOOMINGTON, IN (US)

(21) Appl. No.: 15/332,363

(22) Filed: Oct. 24, 2016

Related U.S. Application Data
(60) Provisional application No. 62/254,256, filed on Nov. 12, 2015.

(51) Int. Cl. A61M 39/02; A61M 25/09

(52) U.S. Cl. CPC A61M 39/0247; A61M 25/09; A61M 2039/0258; A61M 2039/0264; A61M 2039/0261; A61M 2039/027; A61M 2039/0202; A61M 2039/0273

(57) ABSTRACT

The present invention generally relates to device and system for facilitating vascular access into a vessel of a patient and to a method of using such a device or system. In one embodiment, the vessel is a vein and the device is used to facilitate obtaining a blood sample.

### RELATED APPLICATIONS

This non-provisional patent application claims priority to U.S. Provisional Patent Application No. 62/254,256, filed Nov. 12, 2015, the contents of which are incorporated by reference in their entirety.

### TECHNICAL FIELD

The present invention generally relates to device and system for facilitating vascular access into a vessel of a patient and to a method of using such a device or system. In one embodiment, the vessel is a vein and the device is used to facilitate obtaining a blood sample.

### BACKGROUND

The Seldinger technique, also known as Seldinger wire technique, is a medical procedure to obtain safe access to vessels, for example blood vessels, or other hollow organs. The desired vessel or cavity is punctured with a sharp hollow needle called a trocar. A round-tipped guidewire is then advanced through the lumen of the trocar, and the trocar is withdrawn. A “sheath” or blunt cannula can now be passed over the guidewire into the cavity or vessel. Alternatively, drainage tubes are passed over the guidewire. After passing a sheath or tube, the guidewire is withdrawn.

A sheath can be used to introduce catheters or other devices to perform endoluminal (inside the hollow organ) procedures, such as angioplasty. Fluoroscopy may be used to confirm the position of the catheter and to maneuver it to the desired location. Injection of radiocontrast may be used to visualize organs. Interventional procedures, such as thermo-ablation, angioplasty, embolization or biopsy, may be performed. Upon completion of the desired procedure, the sheath is withdrawn. In certain settings, a sealing device may be used to close the hole made by the procedure.

Intravenous (IV) cannulation is a technique in which a cannula is placed inside a vein to provide venous access. Venous access allows sampling of blood as well as administration of fluids, medications, parenteral nutrition, chemotherapy, and blood products.

When performing the Seldinger technique to gain vessel access or provide therapy, two significant difficulties may arise. Initially, it may be difficult to find a vessel, for example a vein, that is distended enough to puncture with a needle or other access device. Furthermore, after removal of the access device, it may be difficult to control bleeding from the entry point.

### BRIEF SUMMARY

One aspect of the present invention provides a device for facilitating vascular access into a vessel of a patient. One embodiment of the device includes a housing having an interior chamber and having a peripheral rim defining an opening. A fluid port is positioned in a wall of the housing and provides for fluid ingress and egress to the interior chamber. In addition, a cannula access port is also positioned in the wall of the housing. The device may also include a ring clamp attaching to the peripheral rim. In certain other embodiments, the device includes an adhesive coating on the peripheral rim.

In some embodiments, the device also includes a compliant gasket positioned on the peripheral rim. In other embodiments, the device includes a fastener attached to an exterior of the domed housing and sized and shaped to extend about a body part of the patient and to hold the peripheral rim against the patient. The device may have a viewing window positioned in the wall of the housing. In other embodiments, the housing is formed from a transparent material.

Another aspect of the invention provides a system for facilitating vascular access into a vessel of a patient. One embodiment of the system includes a device including a housing having a peripheral rim defining an opening and an interior chamber. A fluid port is positioned on a wall of the housing and provides fluid ingress and egress to the interior chamber. A cannula access port is also positioned on the wall of housing. The system also includes a pressure/vacuum source that is connectable to the fluid port.

In another embodiment, the system also includes an adhesive-backed tape having a size sufficient to cover the opening. In yet another embodiment, the system includes a ring clamp attaching to the peripheral rim.

Another aspect of the invention provides a method for facilitating vascular access into a vessel of a patient. One embodiment of the method includes placing the opening of a vascular access device as described herein against a body part of the patient and securing the device to the body part so as to position the opening over the vessel.

Once the device is in position, the pressure in the interior chamber is reduced for a time sufficient to distend the vessel and a cannula or other access device inserted through the cannula access port and into the vessel to provide for access to the vessel. After access is achieved, a procedure may be performed, for example, a sample may be obtained from the vessel. The cannula is then withdrawn from the vessel and from the cannula access port. The interior chamber may be subjected to an increased pressure for a time sufficient to constrict the vessel before the device is removed from the body part of the patient.

In one embodiment, the device also includes a ring clamp attaching to the peripheral rim. Securing the device to the body part of the patient includes attaching the adhesive side of an adhesive-backed tape to the body part and securing the adhesive-backed tape with the ring clamp. In another embodiment, the device includes a fastener attached to the exterior of the housing. Here, securing the device to the body part includes placing the fastener around the body part and tightening the fastener to hold the peripheral rim against the body part.

In various embodiments, accessing the vessel includes withdrawing a blood sample from the vessel through the cannula or introducing a wire guide or a catheter into the vessel. The vessel may be, for example, a vein.

### BRIEF DESCRIPTION OF THE DRAWINGS

FIG. 1 is a schematic illustration showing a cross-sectional side view of one embodiment of a vascular access device.

FIG. 2 is a schematic illustration showing a plan view of one embodiment of a vascular access device.

FIG. 3 is a schematic illustration showing a one embodiment of a vascular access device having an attachment feature.

FIG. 4 is a schematic illustration showing a cross-sectional view of a vascular access device positioned to allow access to a vessel.

FIGS. 5(a)-(d) illustrate one embodiment of a method of accessing a vessel using a vascular access device.

FIGS. 6(a)-(f) illustrate another embodiment of a method of accessing a vessel using a vascular access device.

FIGS. 7(a)-(b) illustrate another embodiment of a method of positioning a vascular access device to allow for accessing a vessel.

### DETAILED DESCRIPTION

For the purpose of promoting an understanding of the principles of the invention, reference will now be made to embodiments, some of which are illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Each disclosed feature or features can be combined with the generalized features discussed herein, to form a disclosed embodiment of the present invention.

One aspect of the present invention provides a device for facilitating vascular access into a vessel, for example, an artery or a vein, of a human or veterinary patient. The vascular access device may assist in obtaining access into the vessel by helping to distend the vessel enough to allow for its puncture with a needle or other puncture device. The device may also assist in the constriction of the vessel and the control of bleeding from the entry point after vascular access is complete and the puncture device is removed. The vessel may be any vessel, typically an artery or a vein close to the surface of any part of the patient's body (“body part”.) In certain preferred embodiments, the vessel is a vein close to the surface of a limb of the patient.

In operation, the device allows for the surface overlaying the vessel to be subjected to a reduced pressure for a time sufficient to distend the vessel and hence provide for easier access to the vessel with a puncture device. The device may also assist in the closure of the access puncture site in the vessel by allowing the surface overlaying the vessel to be subjected to an increased pressure for a time sufficient to constrict the vessel, in particular, access puncture site.

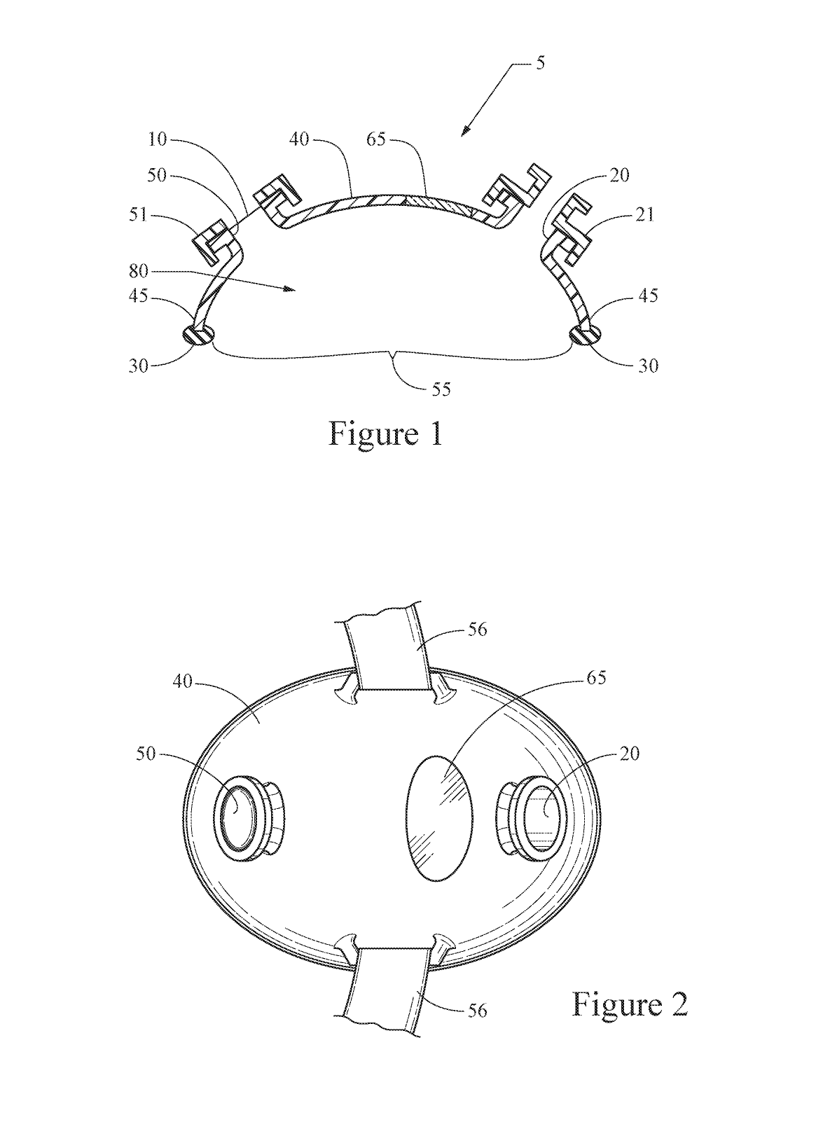

Turning now to FIG. 1, there is shown a schematic illustration of a cross sectional view one embodiment of a vascular access device of the present invention. Vascular access device 5 includes housing 40 having an interior chamber 80 and a peripheral rim 45 defining an opening 55. At least one fluid port 20 is positioned in the wall of housing 40 and provides for fluid ingress and egress to interior chamber 80. In a preferred embodiment, fluid port 20 allows for air to be removed from for added to the interior chamber. At least one cannula access port 50 is also positioned in the wall of housing 40 and may include membrane 10, through which a cannula device such as a trocar, needle, or other puncture device may be passed. In one embodiment, such as that illustrated in FIG. 1, fluid port 20 includes a collar 21 and/or cannula access port 50 includes a collar 51. The collar(s) may assist in retention of membrane 10 in cannula access port 50 and/or provide for the connection of a vacuum/pressure source to fluid port 20. In other embodiments, the ports do not include separate collars.

Membrane 10 may be a self-sealing membrane allowing for the passage of the device through the port, while preventing the passage of gasses and/or liquid. Alternatively, the cannula access port may include a pneumostatic valve. Hence, the pressure at interior chamber 80 of the device may be maintained at a raised or lowered level with respect to the air pressure outside at the exterior of the device when opening 55 is positioned against the skin of a patient so as to form a fluid seal and the cannula device is passed through the port.

Housing 40 may include a viewing window 65 having sufficient transparency to allow the operator to observe the interior chamber, and the surface of the patient enclosed by the device, when the device is positioned against the skin of a patient. In other embodiments, all or part of housing 40 is formed from a material having sufficient transparency to allow the operator of the device to observe the interior of the device when it is positioned against the skin of the patient. For example, all or part of the housing may be formed from a transparent or translucent plastic material.

Compliant gasket 30 may be positioned around at least a portion of peripheral rim 45 of housing 40 and may provide for improved sealing of the rim against the skin of the patient. In certain embodiments, an adhesive coating may be applied to peripheral rim 45 or to gasket 30, if present, to assist in maintaining the device in position.

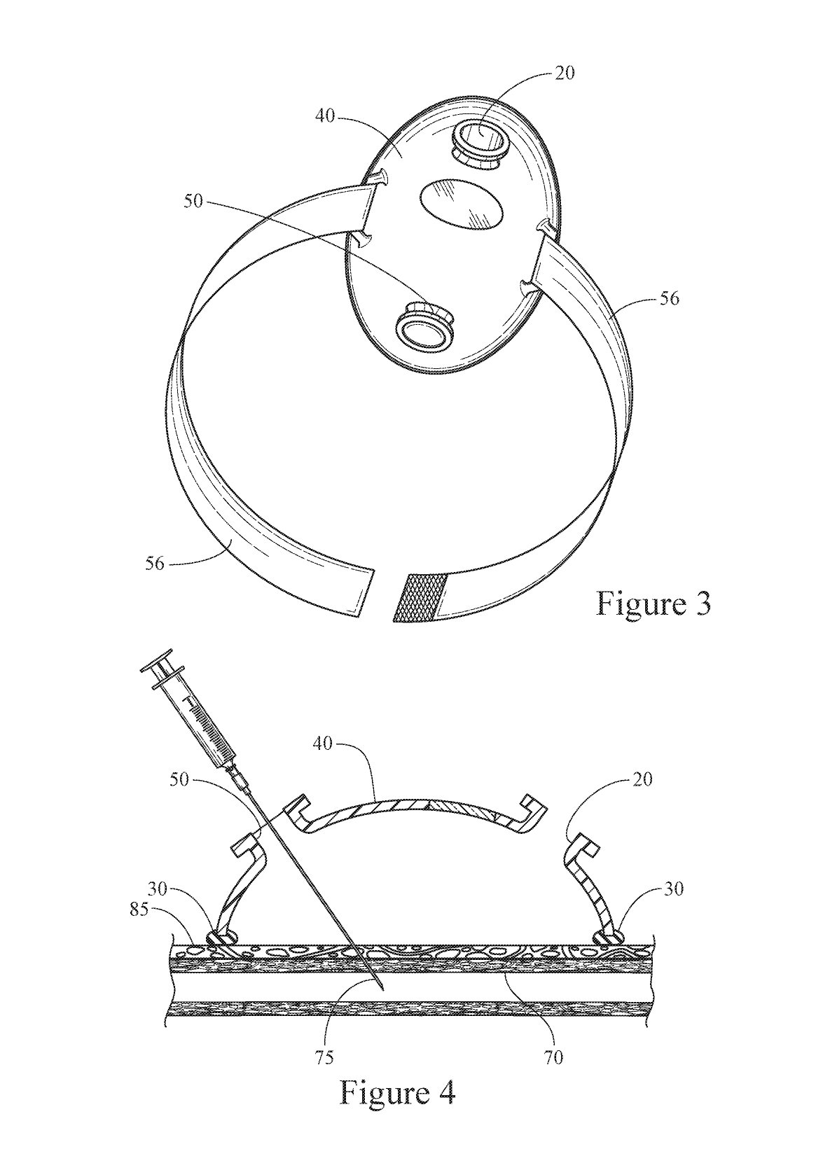

Turning how to FIG. 2, there is shown a plan view of one embodiment of the vascular access device. Housing 40 is shown to include fluid port 20, cannula access port 50 and viewing window 65. Here, housing 40 is shown to have an oval base. However, other housing base shapes are also within the scope of the claimed device, including circular, square, rectangular and irregular shaped bases. All that is required is that the base is shaped to allow access to the puncture site when the device is positioned against the patent to form a fluid seal.

In those embodiments where the device is intended to be placed against the arm and/or leg of the patient, the base of the housing may have a curved or arched profile so as to match the contour of the limb of the patient. For example, in certain embodiments where the device is to be used to obtain and close access to the femoral vessels, the base of the housing may have an irregular shape in order to make a “seal” with specifically the left (or the right) groin area.

Typically, the device is sized such that the cross-sectional dimension of opening 55 is between, for example, 4 cm and 15 cm, or between 5 cm and 12 cm, or between 6 cm and 10 cm. The housing may be formed form any suitable material, for example a plastic material, and may be manufactured by, for example, a molding or vacuum-forming process. Preferably, the device is a disposable single use device. In one embodiment, the device housing is formed from a biodegradable plastic material.

Similarly, although FIG. 1 illustrates a housing having a dome shaped profile, other housing profile shapes, including housings having an irregular shape, are within the scope of the present invention. All that is required is that the interior of the housing has a sufficient volume allow the device to form a seal against the body of the patient so that a sufficiently reduced pressure may be maintained to expand of an underlying part of the vessel of the patient to an extent sufficient to assist in obtaining access to the vessel.

The housing may be rigid, or at least semi-rigid, having sufficiently rigidity to withstand collapse or puncture when the pressure of the interior of the housing is raised or lowered with respect to the air pressure at the outside exterior of the device. In one embodiment, the housing is sufficiently rigid to allow the pressure of the interior of the device to be raised or lowered by between 30 and 60 mm/Hg when the device is positioned against the surface of the patient's body to obtain a fluid seal at opening 55. In another embodiment, the housing may be rigid enough to withstand the vacuum/increased pressure extremes applied during the vascular procedure but flexible enough to allow the housing perimeter to confirm to the contours of the body of the patient.

In certain embodiments, the vascular access device may also include a fastener that assists in holding the device in place against the body of the patient. This may be particularly important when the internal pressure within chamber 80 is raised to constrict the vessel after the access procedure is complete and the operator wishes to constrict the puncture point. FIG. 2 illustrates a portion of fastener 56 attaching to the exterior of housing 40. Fastener 56 is sized and shaped to extend about a limb or other body part of the patient so as to assist in holding the peripheral rim against the patient.

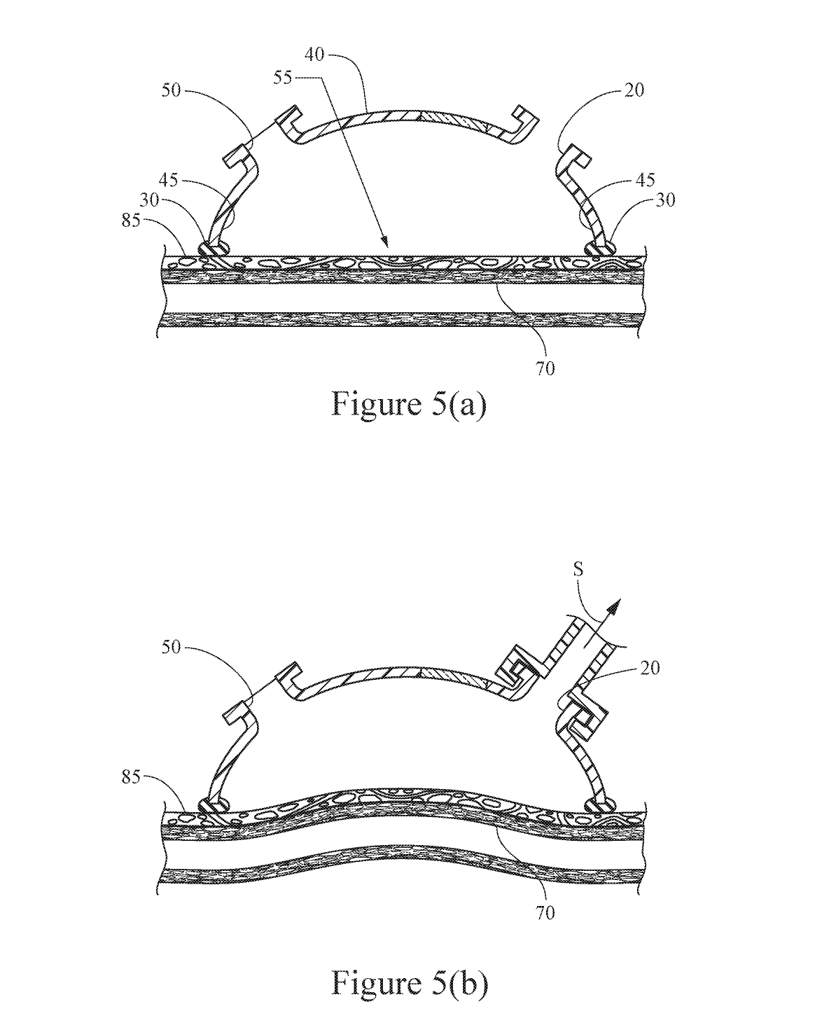

FIG. 3 shows a more complete view of fastener 56. In one embodiment, the dimensions of fastener 56 are such that the fastener extends around a portion of the body of the patient and is then fastened to hold the vascular access device against the patient. For example, the fastener may be sized to extend around arm of the patient to allow for a blood sample to be obtained. In a preferred embodiment, fastener 56 is adjustable to allow the device to be attached to various portions of the body of the patient, such as the arm, the leg or the torso of the patient or to be used with differently sized patients

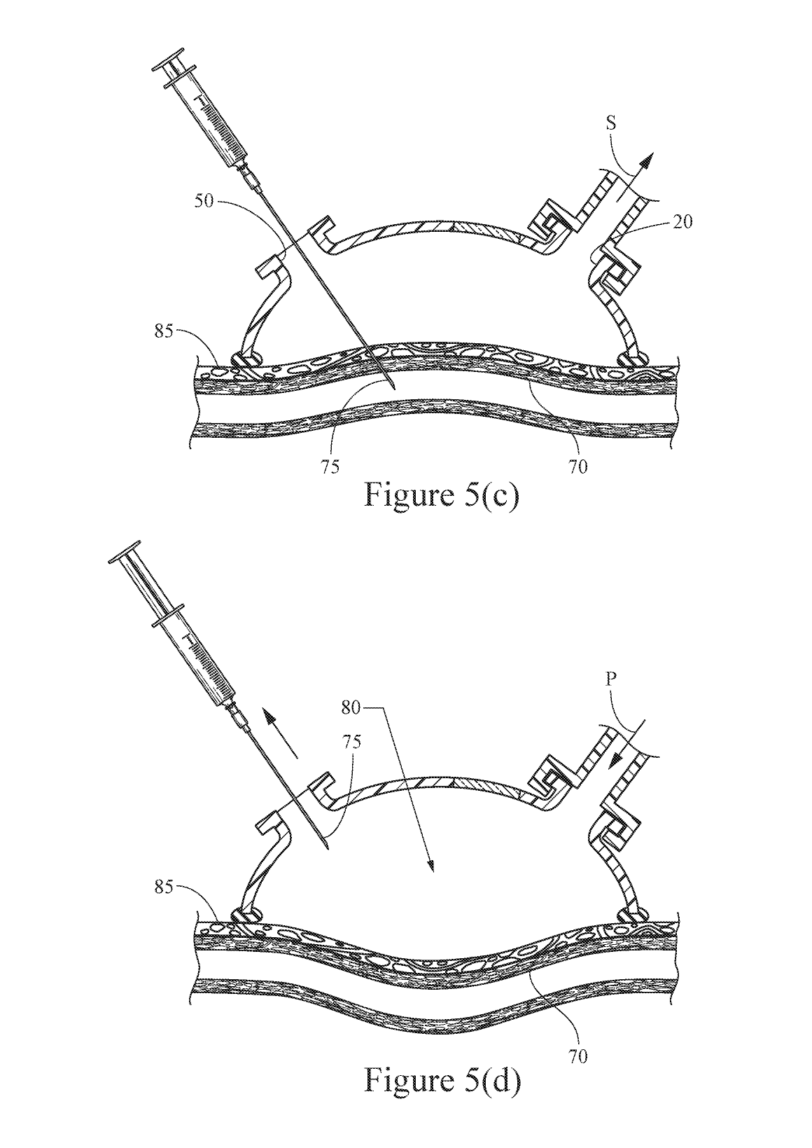

FIG. 4 shows one embodiment of the vascular access device positioned over the surface 85 of a portion of the body of a patient. Needle 75 is shown extending through cannula access port 50 and having its tip inserted through the surface of the patient's skin and into vessel 70. In this figure, the pressure of the interior of the device is maintained at ambient pressure so that the vessel is neither distended nor constricted.

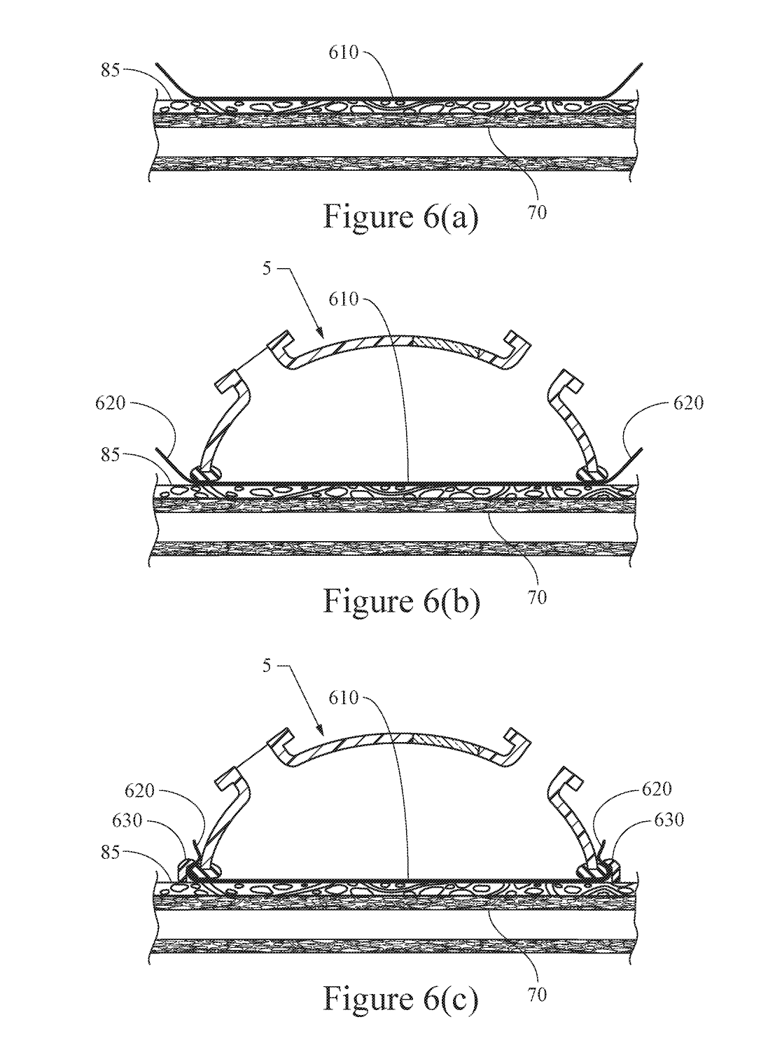

FIGS. 5(a)-5(d) illustrate one embodiment of a method of using the vascular access device to assist in access to the patient vessel and in the closure of the access puncture in the vessel after the procedure is performed. FIG. 5(a) provides a cross-sectional view of chamber 40 positioned against surface 85 of a portion of the body of a patient so as to present opening 55 to the surface.

After the device is positioned and a seal between peripheral rim 45 or gasket 30, if present, and the patient's body is obtained, a vacuum source is applied to fluid port 20 to draw air from the interior of the device. FIG. 5(b) illustrates that vessel 70 underlying patient surface 85 is distended as a result of the reduction in the pressure of the interior of the device (Suction “S”) , in particular, the region overlying the vessel. This distension extends the vessel and makes the vessel 70 more accessible for puncture to allow access to the vessel. The operator may observe the distended vessel through a viewing window in the device housing, or, alternatively, through the housing itself if the housing is formed from a transparent or translucent material.

Turning now to FIG. 5(c), needle 75, or other puncture device, is inserted through cannula access port 50 and positioned so that its tip punctures the vessel wall and enters the vessel. Cannula access port may be any port that allows for access of the puncture device to the interior chamber while not permitting fluid flow through the port. For example, as is disclosed above, cannula access port 50 may include a self-sealing membrane or a pneumostatic valve. Once access to the interior of the vessel is achieved, the procedure requiring access to the vessel may be performed. For example, if the vessel is a vein, a blood sample may be obtained.

In other embodiments of the method, a wire guide or a similar device many be inserted into the vessel to allow another procedure to be performed. In such embodiments, the device may be removed after access to the vessel is obtained and later re-attached and put under increased pressure to assist in sealing the access puncture after the wire guide or other device is removed.

As illustrated in FIG. 5(d), after access to the vessel is obtained and any required procedure completed, the internal pressure within chamber 80 is raised (Pressure “P”) to constrict vessel 70 and close the puncture so as to reduce or prevent any leakage from the vessel after needle 75 or other puncture device is removed from the vessel. Needle 75 or similar device may be removed from the vessel and/or from cannula access port 50 before or after the pressure within the chamber of the device is increased.

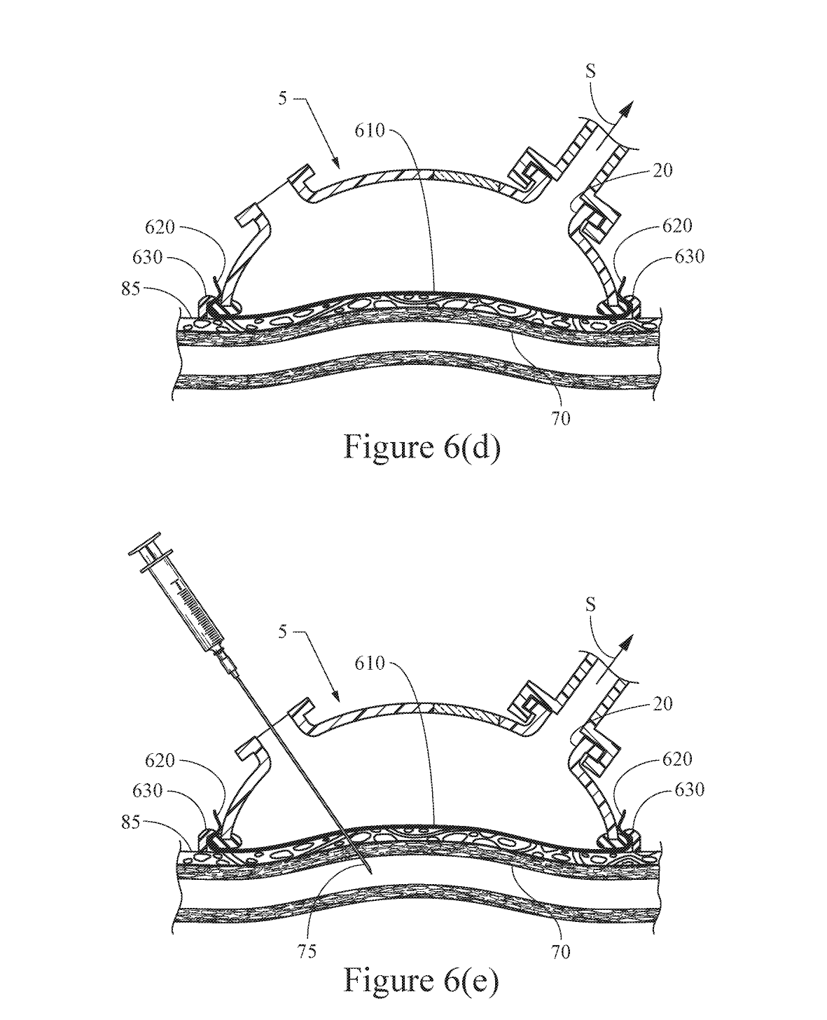

FIGS. 6(a)-(f) illustrate another embodiment of a method of using the vascular access device to assist in access to a vessel and in the closure of the access puncture point in the vessel after a procedure requiring access is performed. Turning to FIG. 6(a), adhesive-backed tape or film 610 is placed over surface 85 of the body of the patent, above vessel 70. Here, at least the side of the tape contacting surface 85 includes an adhesive coating, enabling the attachment of the tape to surface 85 of a body part of the patient.

FIG. 6(b) illustrates vascular access device 5 positioned on top of adhesive backed tape 610. Perimeter portions 620 of adhesive-backed tape 610 are then attached to vascular access device 5 to secure the device to the tape and hence to the surface. For example, FIG. 6(c) illustrates the attachment of adhesive-backed tape to vascular access device 5 by wrapping portions of the perimeter of the tape around the perimeter of the device and fixing the tape in position by ring clamp 630. However, other methods may be used to attach the device to the adhesive-backed tape. For example, the top surface of the tape may also include an adhesive coating and/or the peripheral rim 45, or gasket 30, of the vascular access device may include an adhesive coating.

In any case, after the device is attached to the adhesive-backed tape, a vacuum source is applied to fluid port 20 to draw air from the interior chamber of the device, as is illustrated in FIG. 6(d), resulting in the distension of vessel 70 underlying patient surface 85. After vessel is distended, needle 75, or other puncture device, is inserted through cannula access port 50 and positioned so that its tip punctures the vessel wall and enters the vessel. See FIG. 6(e). After access is achieved and any required procedure performed, a pressure source is applied to fluid port 20 in the internal pressure within the chamber of the device is increased , as illustrated in FIG. 6(f), resulting in the constriction of vessel 70 and the sealing of the puncture point from the needle, or other puncture device. Vascular access device Sand adhesive-backed tape 610 may then be removed from the surface of the patient's body.

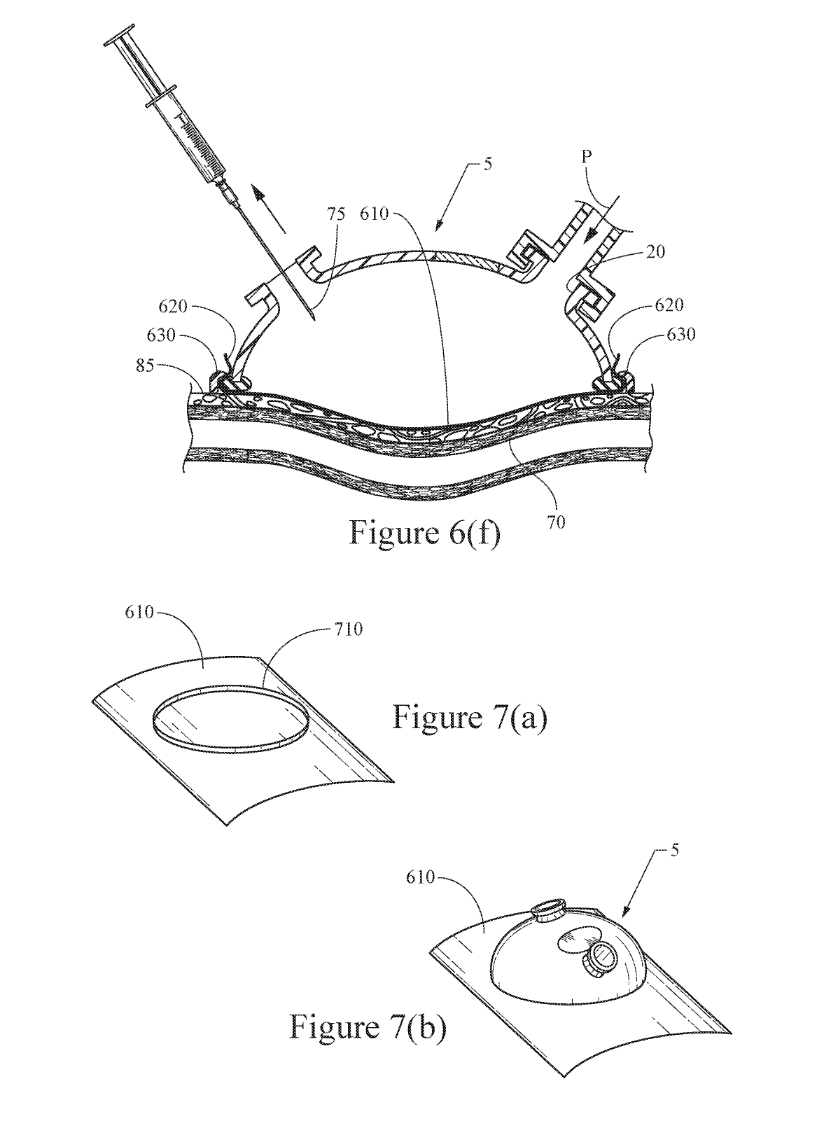

Turning now to FIGS. 7(a)-(b), there is illustrated another embodiment of adhesive-backed tape 610. Here, as is illustrated in FIG. 7(a), the top surface of the tape includes rim 710, which is sized and shaped to accept vascular access device 5 so that rim 710 is positioned against the perimeter of peripheral rim 45, or gasket 30. Rim 710 may assist in the positioning of the device on the surface of the patient's body and may also assist in the attachment of the device to the tape. For example, rim 710 may be provided with an adhesive coating.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

I claim:

1. A device for facilitating vascular access into a vessel of a patient, the device comprising
a housing having an interior chamber and comprising a peripheral rim defining an opening;
a fluid port positioned in a wall of the housing and providing for fluid ingress and egress to the interior chamber;
a cannula access port positioned in the wall of the housing; and
a ring clamp attaching to the peripheral rim.

2. The device of claim 1, further comprising a compliant gasket positioned on the peripheral rim.

3. The device of claim 1, further comprising a fastener attaching to an exterior of the domed housing; wherein the fastener is sized and shaped to extend about a limb of the patient and to hold the peripheral rim against the limb.

4. The device of claim 1, further comprising a viewing window positioned in the wall of the housing.

5. The device of claim 1, wherein the housing is formed from a transparent material.

6. The device of claim 1, wherein the cannula access port comprises a pneumostatic valve.

7. The device of claim 1, wherein the housing is sufficiently rigid to withstand collapse under a vacuum pressure of up to 60 mm Hg applied to the interior of the housing.

8. The device of claim 1, wherein the housing is a domed shaped housing and wherein the opening forms a base of the dome.

9. The device of claim 8, wherein the opening is an oval opening.

10. The device of claim 1, further comprising an adhesive coating on the peripheral rim.

11. The device of claim 1, wherein housing comprises a plastic material.

12. A system for facilitating vascular access into a vessel of a patient, the system comprising:
(a) a device comprising:
a housing having an interior chamber and comprising a peripheral rim defining an opening;
a fluid port positioned in a wall of the housing and providing fluid ingress and egress to the interior chamber; and
a cannula access port positioned on the wall of housing, and
(b) a pressure/vacuum source; wherein the pressure/vacuum source is connectable to the fluid port.

13. The system of claim 12, further comprising an adhesive-backed tape.

14. The system of claim 12, wherein the device further comprises a ring clamp attaching to the peripheral rim.

15. A method for facilitating vascular access into a vessel of a patient, the method comprising:
placing an opening of a vascular access device against a body part of the patient,
wherein the device comprises:
a housing having an interior chamber and comprising a peripheral rim defining an opening;
a fluid port positioned in a wall of the housing and providing fluid ingress and egress to the interior chamber; and
a cannula access port positioned in the wall;
securing the device to the body part so as to position the opening over the vessel;
subjecting the interior chamber to a reduced pressure for a time sufficient to distend the vessel;
inserting a puncture device through the cannula access port and into the vessel;
accessing the vessel through the puncture device;
withdrawing the puncture device from the vessel and the cannula access port;
subjecting the interior chamber to an increased pressure for a time sufficient to constrict the vessel; and
removing the vascular access device from the body part of the patient.

16. The method of claim 15, wherein the device further comprises a ring clamp attaching to the peripheral rim and wherein securing the device to the body part comprises:
attaching an adhesive-backed tape to the body part, wherein a side having an adhesive coating is attached to the body part ; and
securing the adhesive-backed tape to the device by the ring clamp.

17. The method of claim 15, wherein the device further comprises a fastener attaching to an exterior of the housing; wherein the fastener is sized and shaped to extend about the patient and wherein securing the device to the body part comprises placing the fastener around the patient and tightening the fastener to hold the peripheral rim against the body part.

18. The method of claim 15, wherein accessing the vessel through the puncture device comprises withdrawing a blood sample from the vessel through the puncture device.

19. The method of claim 15, wherein accessing the vessel through the puncture device comprises introducing a wire guide or a catheter into the vessel.

20. The method of claim 15, wherein the vessel is a vein.
